(19) United States
(12) Patent Application Publication
(10) Pub. No.: US 2026/0216310 A1
(43) Pub. Date: Jul. 30, 2026

(54) METHODS OF VACCINE ADMINISTRATION TO SALMONIDS

(71) Applicant: Zoetis Services LLC, Parsippany, NJ (US)

(72) Inventors: Adérito Luis MONJANE, Oslo (NO); Marius Andre de Feijter KARLSEN, Oslo (NO); Øyvind HAUGLAND, Oslo (NO)

(21) Appl. No.: 19/137,065

(22) PCT Filed: Dec. 13, 2023
(86) PCT No.: PCT/US2023/083710

§ 371 Date: Jun. 9, 2025

Related U.S. Application Data
(60) Provisional application No. 63/378,120, filed on Oct. 3, 2022.

(51) Int. Cl. A61K 39/12; A61K 9/00; A61K 9/107; A61K 39/00

(52) U.S. Cl. CPC A61K 39/12; A61K 9/0019; A61K 9/107; A61K 2039/53; A61K 2039/54; A61K 2039/545; A61K 2039/552; A61K 2039/55522; A61K 2039/70

(57) ABSTRACT

The disclosure provides compositions and methods for salmonid vaccination against PMCV and at least one other pathogen

### FIELD OF THE INVENTION

This invention is generally in the field of vaccinating salmonids.

### BACKGROUND

Cardiomyopathy syndrome (CMS) is a disease affecting primarily large Atlantic salmon in the second year in seawater close to harvest, and therefore the economic impact is significant. Affected fish may suddenly die without showing signs of disease or may show symptoms such as abnormal swimming behavior and anorexia. CMS is diagnosed on the basis of histopathology, showing severe Inflammation and degeneration of spongious myocardium in the atrium and ventricle. A possible secondary effect of circulatory disturbance is multifocal liver necrosis which is commonly observed.

The infectious nature of CMS was finally demonstrated by the isolation and cultivation of a virus causing CMS in fish as reported in the Norwegian patent application NO 2008 2869. The virus was named Piscine Myocarditis Virus (PMCV). It is characterized by the ability of introducing one or more symptoms selected from the group consisting of: skin haemorrhages, raised scales, exopthalamos, ascites, fibrinous casts over the liver capsule, blood or blood clots filling the pericardial cavity, ruptures in the cardiac atrial wall, dilation of the cardiac atrium, compression of the cardiac ventricle, inflammation of the spongious myocardium and the epi- and endocardium, liver lesions including multifocal to anastomosing necrosis of hepatocytes and fibrinous coating of the capsule, congestion of the spleen and/or the gills.

The host cells for cultivation of PMCV disclosed in NO 2008 2869 have shown to results in low yield of the virus. There is still a need for further knowledge to be able to develop efficient means for controlling the disease and to be able to develop efficient vaccines, such as recombinant vaccines. To that effect, WO2011/131600 disclosed nucleic acid sequences from PMCV.

DNA vaccines have become an attractive approach for generating antigen-specific immune responses because of their stability and simplicity of delivery. DNA vaccines can be easily prepared in large scale with high purity, repeatedly administered and are highly stable relative to proteins and other biological polymers.

In course of the lifecycle of the salmonids, there is only a limited number of times that the fish can be vaccinated before their mortality and general health are affected, preferably, before the fish is transferred to sea water. At the same time, as more pathogens affecting salmonid health are discovered, it is desirable to vaccinate fish against as many pathogens as possible. Fish vaccination is a relatively labor-intensive operation—for practical reasons it is beneficial to vaccinate against the greatest possible number of antigens at once. Thus, there is a need in the art for the methods of co-administering multiple vaccines to salmonids.

### SUMMARY OF INVENTION

This disclosure provides a DNA vaccine comprising a nucleic acid sequence encoding SEQ ID NO: 3 or SEQ ID NO: 4 or an amino acid sequence at least 90% identical to SEQ ID NO: 3 or SEQ ID NO: 4 for use in protecting a salmonid against PMCV and at least one other pathogen, wherein said DNA vaccine is injected intramuscularly; a second vaccine is injected intraperitoneally substantially simultaneously with the DNA vaccine; said second vaccine comprises an antigen that elicits protective response against said at least one other pathogen; and said second vaccine is formulated as a W/O emulsion.

In certain embodiments, the nucleic acid sequence in the DNA vaccine disclosed herein encodes SEQ ID NO: 5 or an amino acid sequence at least 90% identical thereto. In more preferred embodiments, wherein the nucleic acid sequence encodes SEQ ID NO: 5 or an amino acid sequence at least 95% identical thereto, and wherein further at least 50% of the amino acids different from SEQ ID NO: 5 are conservative substitutions.

The DNA vaccine according to any of the embodiments recited above may further comprise a nucleic acid sequence encoding a molecular adjuvant. In certain embodiments, the molecular adjuvant is IFNb comprising SEQ ID NO: 7 or IFNb1 comprising SEQ ID NO: 9, or a sequence that is at least 95% identical to SEQ ID NO: 7 or SEQ ID NO: 9. Preferably the amino acids that differ from SEQ ID NO: 7 or SEQ ID NO: 9 are conservative substitutions.

The use of the DNA vaccines according to any of the above-referenced embodiments, comprises two or three intramuscular injections, said intramuscular injections administered substantially simultaneously. Preferably, the total dose of the molecular adjuvant in said DNA vaccine administered in two or three injection is no greater than the dose of the molecular adjuvant in said DNA vaccine administered in a single injection. Even more preferably, the total dose of the antigen in said DNA vaccine administered in two or three injection is no greater than as the dose of the antigen in said DNA vaccine administered in a single injection.

The DNA vaccine according to any of the embodiments recited above is administered with a second vaccine, the second vaccine being a water-in-oil emulsion and being administered intraperitoneally. In different embodiments, the second vaccine comprises one or more of the following antigens: Moritella viscosa, Piscirickettsias sp. Aeromonas sp., Vibrio sp., Aliivibrio sp., Listonella sp., Tenacibaculum sp., Pasteurella sp., Photobacterium sp, Flavobacterium sp., Yersinia sp., Renibacterium sp., Streptococcus sp., Lactococcus sp., Leuconostoc sp., Bifidobacterium sp., Pediococcus sp., Brevibacterium sp., Edwarsiella sp., Francisella sp., Pseudomonas sp., Cytophaga sp., Nocardia sp., Mycobacterium sp., Viral Hemorrhagic Septicemia Virus (VHSV), Infectious Hematopoietic Necrosis virus (IHNV), Infectious Pancreatic Necrosis Virus (IPNV), Infectious Salmon Anaemia virus (ISAV), Salmon pancreatic disease virus (SPDV), Iridovirus, Nodavirus, Piscine myocarditis virus (PMCV) and Piscine Orthoreovirus (PRV).

In more particular embodiments, the second vaccine comprises one or more (including all of) the following antigens: IPNV, ISAV, SPDV, Aeromonas salmonicida, Vibrio anguillarum O1, O2, Vibrio (Aliivibrio) salmonicida, Yersinia ruckeri O1 and Moritella viscosa. In other embodiments, the second vaccine comprises one or more (including all of) the following antigens: IPNV, M. viscosa, Aeromonas salmonicida, Vibrio anguillarum serotype 1 and O2, and Vibrio (Aliivibrio) salmonicida.

In certain embodiments, the vaccines as described according to any of the embodiments described above and administered according to any of the embodiments described above, are administered to salmonids selected from the group consisting of Atlantic salmon (Salmo salar), coho salmon (Oncorhynchus kisutch), rainbow trout (Oncorhynchus mykiss), sockeye salmon (Oncorhynchus nerka) and Chinook salmon (Oncorhynchus tshawytscha); and most preferably to Salmo salar.

The DNA vaccines used as described in any of the embodiments above reduce the frequency, the intensity, or the duration of at least one clinical sign selected from the group consisting of mortality, lack of weight gain, skin ulcers, and a heart defect.

In certain embodiments, the DNA vaccines described above are administered to salmonids who are not subjected to a smoltification signal within two weeks of injecting said DNA vaccine, preferably within six weeks. In certain embodiments, the smoltification signal is light, feed or water temperature.

### DETAILED DESCRIPTION

The term “about” as applied to a reference number refers to the reference number plus or minus 10 percent of said value.

The term “different injection sites” refers to injection points located sufficiently apart from each other so that different skin penetrations are made for the injections into these injection points. For example, the injection sites may be at least 0.5 mm apart. In different embodiments, the injection sites are 0.5 to about 100 mm apart or 0.5 to about 50 mm apart from each other, or about 1 to about 40 mm apart, or about 3 to about 30 mm apart or about 5 to about 25 mm apart, or about 5 to about 20 mm apart, or about 10 to about 20 mm apart, or about 10 mm to about 30 mm apart, or about 10 mm to about 40 mm apart, or about 10 mm to about 50 mm apart, or about 10 mm to about 60 mm apart, or about 10 mm to about 70 mm apart, or about 10 mm to about 80 mm apart, or about 10 mm to about 90 mm apart, or about 20 mm to about 30 mm apart, or about 20 mm to about 40 mm apart, or about 20 mm to about 50 mm apart, or about 20 mm to about 70 mm apart, or about 30 mm to about 40 mm apart.

The term “effective dose” refers to the amount of the antigen which, if administered as a single injection in a given formulation, would provide the desired level of protection against the intended pathogen.

The term “lack of weight gain” refers to the inability of the infected non-vaccinated fish to gain weight on par with non-infected fish. More specifically, it refers to the difference of at least 5%, and more preferably 10%, or 15%, or 20% in weight between the non-infected fish and infected non-vaccinated fish, wherein the weight difference of at least 5% (compared to the non-infected fish) persists for at least four weeks or longer, e.g. at least five weeks or at least six weeks.

The term “No greater than” means equal to or less than the reference value, with the allowance of the measuring error of the injection system.

The terms “protect”, “protection” and the like refer to the ability of the vaccines disclosed herein to reduce or eliminate the duration or the severity of at least one clinical sign of the pathogen against which the salmonids are vaccinated. The clinical signs vary depending on the pathogen and include, without limitations, heart defects, skeletal muscle defects, skin defects, lack of weight gain, and mortality. Clinical signs also include reduced viral counts in the relevant organs as well as reduced ability to infect other salmonids.

The term “substantially simultaneously” refers to the timing of administering multiple injections. The injections are administered substantially simultaneously, if all of these multiple injections are administered within 5 minutes (from the first injection to the last injection), preferably within 4 minutes, or within three minutes, or within 2 minutes, or within 1 minute or within 30 seconds or within 15 seconds.

In a broad aspect, the invention provides A DNA vaccine comprising a nucleic acid sequence encoding a PMCV protein or a fragment thereof that is capable of eliciting protective immune response for use in a method of protecting a salmonid against PMCV and at least one other pathogen, wherein: said DNA vaccine is injected intramuscularly, a second vaccine is injected intraperitoneally substantially simultaneously with the DNA vaccine, said second vaccine comprises an antigen that elicits protective response against said at least one other pathogen; and said second vaccine is formulated as a W/O emulsion.

### PMCV DNA Vaccine

The PMCV DNA vaccines disclosed herein comprise a nucleic acid sequence that encode a PMCV protein or a fragment thereof that is capable of eliciting protective immune response. In certain embodiments, the PMCV protein is encoded by PMCV ORF-1 and comprises the protein according to SEQ ID NO: 3 or a sequence that is at least 90% identical to SEQ ID NO: 3 (e.g., at least 91%, or at least 92% or at least 93% or at least 94%, or at least 95% or at least 97%, or at least 98% or at least 99% or 100% identical to SEQ ID NO: 3). If the PMCV protein is less than 100% identical to SEQ ID NO: 3, then preferably, the mutations leading to the differences from SEQ ID NO: 3 are substitutions. In certain advantageous embodiments, at least half (or at least 60% or at least 70% or at least 80%, or at least 90% or 100%) of differing amino acids are conservative substitutions.

In other embodiments, the PMCV protein comprises an internal deletion such that the resulting protein is at least 95% identical to SEQ ID NO: 4 (e.g., at least 91%, or at least 92% or at least 93% or at least 94%, or at least 95% or at least 97%, or at least 98% or at least 99% or 100% identical to SEQ ID NO: 4). If the PMCV protein is less than 100% identical to SEQ ID NO: 4, then preferably, the mutations leading to the differences from SEQ ID NO: 4 are substitutions. In certain advantageous embodiments, at least half (or at least 60% or at least 70% or at least 80%, or at least 90% or 100%) of differing amino acids are conservative substitutions.

In other embodiments, the antigen in the PMCV DNA vaccine is provided as a fusion protein, comprising, from the 5′ to 3′:
a) a N-terminal secretion signal sequence of a secreted or a first membrane-bound protein;
b) the PMCV protein or the fragment thereof, as described in any of the embodiments recited above;
c) a transmembrane domain of a second membrane-bound protein.

In certain embodiments, the first and the second membrane proteins are identical. Preferably, the first (and the second) protein is viral hemorrhagic septicemia virus G-protein (VHSV-G).

Thus, in some embodiments, said N-terminal secretion signal sequence is at least 90%, or at least 91%, or at least 92%, or at least 93%, or at least 94%, or at least 95%, or at least 96%, or at least 97%, or at least 98%, or at least 99%, or 100% identical to SEQ ID NO: 1 or SEQ ID NO: 11. If the N-terminal secretion signal sequence is less than 100% identical to SEQ ID NO: 1 or SEQ ID NO: 11, then preferably, the mutations leading to the differences from SEQ ID NO: 1 or SEQ ID NO: 11 are substitutions. In certain advantageous embodiments, at least half (or at least 60% or at least 70% or at least 80%, or at least 90% or 100%) of differing amino acids in said N-terminal secretion signal are conservative substitutions.

In certain embodiments, said transmembrane domain is at least 90%, or at least 91%, or at least 92%, or at least 93%, or at least 94%, or at least 95%, or at least 96%, or at least 97%, or at least 98%, or at least 99%, or 100% identical to SEQ ID NO: 2 or SEQ ID NO: 12. If the transmembrane sequence is less than 100% identical to SEQ ID NO: 2 or SEQ ID NO: 12, then preferably, the mutations leading to the differences from SEQ ID NO: 2 or SEQ ID NO: 12 are substitutions. In certain advantageous embodiments, at least half (or at least 60% or at least 70% or at least 80%, or at least 90% or 100%) of differing amino acids in said transmembrane are conservative substitutions.

In certain preferred embodiment, the fusion protein comprises SEQ ID NO: 5 or an amino acid sequence that is at least 90% identical thereto. In different embodiments, the amino acid sequence is at least 91%, or at least 92%, or at least 93%, or at least 94%, or at least 95%, or at least 96%, or at least 97%, or at least 98%, or at least 99%, or 100% identical to SEQ ID NO: 5. If the fusion protein is less than 100% identical to SEQ ID NO: 5, then preferably, the mutations leading to the differences from SEQ ID NO: 5 are substitutions. In certain advantageous embodiments, at least half (or at least 60% or at least 70% or at least 80%, or at least 90% or 100%) of differing amino acids in said fusion protein are conservative substitutions.

### Molecular Adjuvant

The DNA vaccine according to any of the embodiments recited herein may further contain a nucleic acid sequence encoding a molecular adjuvant. In certain embodiment, the molecular adjuvant is an interferon. In particularly preferred embodiments, the interferon is IFNb or IFNb1.

Interferon IFNb protein comprises SEQ ID NO: 7 or an amino acid sequence that is at least 90% identical thereto. In different embodiments, the amino acid sequence is at least 91%, or at least 92%, or at least 93%, or at least 94%, or at least 95%, or at least 96%, or at least 97%, or at least 98%, or at least 99%, or 100% identical to SEQ ID NO: 7. If the IFNb is less than 100% identical to SEQ ID NO: 7, then preferably, the mutations leading to the differences from SEQ ID NO: 7 are substitutions. In certain advantageous embodiments, at least half (or at least 60% or at least 70% or at least 80%, or at least 90% or 100%) of differing amino acids in said IFNb protein are conservative substitutions.

Interferon IFNb1 comprises SEQ ID NO: 9 or an amino acid sequence that is at least 90% identical thereto. In different embodiments, the amino acid sequence is at least 91%, or at least 92%, or at least 93%, or at least 94%, or at least 95%, or at least 96%, or at least 97%, or at least 98%, or at least 99%, or 100% identical to SEQ ID NO: 9. If the IFNb1 is less than 100% identical to SEQ ID NO: 9, then preferably, the mutations leading to the differences from SEQ ID NO: 9 are substitutions. In certain advantageous embodiments, at least half (or at least 60% or at least 70% or at least 80%, or at least 90% or 100%) of differing amino acids in said IFNb1 protein are conservative substitutions.

The skilled person will further acknowledge that alterations of the nucleic acid sequence resulting in modifications of the amino acid sequence of the protein it codes may have little, if any, effect on the resulting three-dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine(S), Threonine (T); [2] Aspartic acid (D), Glutamic acid (E); [3] Asparagine (N), Glutamine (Q); [4] Arginine (R), Lysine (K), Histidine (H); [5] Isoleucine (I), Leucine (L), Methionine (M), Valine (V); and [6] Phenylalanine (F), Tyrosine (Y), Tryptophan (W), (see, e.g., US Patent Publication 20100291549).

In certain embodiments, at least 50%, or at least 60%, or at least 70% or at least 80% or at least 90% or at least 95% or all 100% of amino acids differing from the reference sequence are conservative substitutions.

Protein and/or nucleic acid sequence identities according to any of the embodiments described herein can be evaluated using any of the variety of sequence comparison algorithms and programs known in the art. For sequence comparison, typically one sequence acts as a reference sequence (e.g., a sequence disclosed herein), to which test sequences are compared. A sequence comparison algorithm then calculates the percent sequence identities for the test sequences relative to the reference sequence, based on the program parameters.

The percent identity of two amino acid or two nucleic acid sequences can be determined for example by comparing sequence information using the computer program GAP, i.e., Genetics Computer Group (GCG; Madison, WI) Wisconsin package version 10.0 program, GAP (Devereux et al. (1984), Nucleic Acids Res. 12:387-95). In calculating percent identity, the sequences being compared are typically aligned in a way that gives the largest match between the sequences. The preferred default parameters for the GAP program include: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14:6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps.

Sequence identity and/or similarity can also be determined by using the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (BESTFIT, FASTA, and TFASTA in the Wisconsin Genetics Software Package, Genetics Computer Group, 575 Science Drive, Madison, Wis.).

Another example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xᵤ set to 16, and X_{g} set to 40 for database search stage and to 67 for the output stage of the algorithms. Gapped alignments are triggered by a score corresponding to about 22 bits.

### Expression Cassettes and Vectors

Nucleic acid sequences encoding the antigens of the invention can be designed by a person of ordinary skill in the art based on the amino acid sequences for the antigens described above. In certain embodiments, the nucleic acid sequence encoding the PCMV antigen comprises SEQ ID NO: 6.

The nucleic acid sequence encoding the antigen is subcloned into an expression cassette and is under operable control of a first promoter. The first promoter may be selected from such exemplary promoters as simian virus 40 early promoter (SV40), cytomegalovirus immediate-early promoter (CMV), human Ubiquitin C promoter (UBC), human elongation factor 1a promoter (EF1A), mouse phosphoglycerate kinase 1 promoter (PGK), and chicken β-Actin promoter coupled with CMV early enhancer (CAGG). Generally, the expression cassette also comprises a polyadenylation signal that terminates transcription of the nucleic acid sequence encoding the antigen.

Optionally, the cassette may also comprise a nucleic acid sequence encoding the molecular adjuvant under operative control of a second promoter. In certain preferred embodiments, the nucleic acid encoding SEQ ID NO: 7 comprises SEQ ID NO: 8 and the nucleic acid encoding SEQ ID NO: 9 comprises SEQ ID NO: 10.

Generally, the second promoter should be able to initiate transcription in the host organism. In the embodiments where the host is a salmonid, such as Salmo salar, suitable promoters include, without limitations, simian virus 40 early promoter (SV40), cytomegalovirus immediate-early promoter (CMV), human Ubiquitin C promoter (UBC), human elongation factor 1α promoter (EF1A), mouse phosphoglycerate kinase 1 promoter (PGK), and chicken β-Actin promoter coupled with CMV early enhancer (CAGG). Generally, the expression cassette of the invention also comprises a polyadenylation signal that terminates transcription of the nucleic acid sequence of the invention.

The expression cassette according to any of the embodiments described above may be subcloned into a vector. Different vectors suitable for the invention are known in the art, including both plasmid vectors and viral vectors. Suitable plasmids include, without limitations pUC-based vectors, pVAX-vectors, pcDNA-vectors, NTC-vectors. In a set of preferred embodiments, the vector is NTC9385R (Nature Technology Corporation) or a variant thereof, as described in the Examples.

In other embodiments, a relatively new “doggybone” or dbDNA™ plasmid may be used as a vector. dbDNA™ plasmids as well as the process of making these plasmids have been described at least in WO2018033730, WO2016034849, WO2019193361, WO2012017210 and WO2021161051. The advantage of this approach is that the vector can be synthesized in a cell-free process thus improving manufacturing efficiency. The cell-free process preferably involves amplification of the template via strand displacement replication. This synthesis releases a single stranded DNA, which may in turn be copied into double stranded-DNA, using a polymerase. Alternatively, strand displacement can be achieved by supplying a DNA polymerase and a separate helicase. Replicative helicases may open the duplex DNA and facilitate the advancement of the leading-strand polymerase. The resulting double-stranded DNA concatemer is enzymatically cut and ligated thus forming the doggybone-like shape DNA construct.

Suitable viral vectors include, without limitations, alphaviruses such as Salmonid Alphavirus (SAV), also known as Salmon Pancreatic Disease Virus (SPDV) SAV, rhabdoviruses such as Viral Hemorrhagic Septicemia Virus (VHSV) and Infectious Hematopoietic Necrosis Virus (IHNV), paramyxoviruses such as A Atlantic salmon paramyxovirus (ASPV), adenoviruses, poxviruses such as Salmon gill poxvirus, and the like. These viruses can be genetically modified to remove the parts of the viral genomes responsible for replication. Thus, the resulting viruses would be infectious to fish cells and suitable for production of the antigen, but not be pathogenic.

Preferably the same vector carries both the antigen and, if needed, the molecular adjuvant. However, two different vector molecules may be provided where one molecule carries the antigen and the other carries the molecular adjuvant. Then, if needed, these vector molecules may be combined at a desirable ratio, whether at the stage of the manufacturing or before use.

One of ordinary skill in the art would appreciate that the differences between the described amino acid sequences and the reference amino acid sequences (whether in the context of the fusion protein, or the molecular adjuvant) may be in the form of insertions, deletions, or substitutions. Preferably, the mutations are substitutions, and more preferably, at least some of these substitutions are conservative substitutions.

A person of ordinary skill in the art will appreciate that multiple methods exist for making the amino acid sequences, the nucleic acid sequences, the expression cassettes, and the vectors of disclosed herein. For example, the nucleic acid sequences may be designed using software tools, e.g., CLC Main Workbench, and synthesized artificially or generated using targeted mutagenesis. These sequences may be subcloned into expression cassettes and vectors using genetic engineering techniques widely available to one skilled in the art. See, e.g., Molecular cloning: a laboratory manual (Sambrook & Russell: 2000, Cold Spring Harbor Laboratory Press; ISBN: 0879695773), and: Current protocols in molecular biology (Ausubel et al., 1988+ updates, Greene Publishing Assoc., New York; ISBN: 0471625949).

### Multi-Injection Vaccine Administration

It has previously been discovered (See, e.g. U.S. provisional application 63/375,285 entitled “Method of administration of aquaculture vaccines” and filed on Sep. 12, 2022) that PMCV and PD DNA vaccines administered in several doses are more effective than the same amount of the same DNA vaccines administered in the single dose. Accordingly, in certain embodiments, the DNA vaccine according to the invention is split into two or three portions and delivered in two or three injections, respectively, into different injection sites, said two or three injections administered substantially simultaneously. Preferably, the last injection of the DNA vaccine is administered no later than 4 minutes, no later than 3 minutes, no later than 2 minutes, no later than 1 minute, no later than 45 seconds, no later than 30 second, and no later than 15 seconds, and no later than 5 seconds, and no later than 1 second from the first injection.

The volumes of the DNA vaccine for each of the injections (whether the vaccine is delivered as a single injection or as two or three injections) may be determined by a person of the ordinary skill in the art, but in general, the volumes may be independently chosen from 0.01 ml to about 0.25 ml, and may include about 0.02 ml, about 0.025 ml, about 0.05 ml, about 0.075 ml, about 0.1 ml, about 0.15 ml, about 0.2 ml, or 0.25 ml. It is currently preferred that the members of the plurality of injections should contain substantially the same amount of the antigen.

Multiple methods exist to administer the injections substantially simultaneously. In certain embodiments, commercially available fish vaccination equipment may be fitted with a multi-needle injection tip, where the tips of the needles are configured to be at a desired distance from each other. Suitable vaccination machines include NFT lines of products (Pharmaq) and the specific models include NFT 20, NFT 25 and NFT 30. NFT 20, NFT 25 deliver vaccines intraperitoneally but can be reconfigured for intramuscular injections also. NFT 30 has a special DNA module that is capable of delivering a DNA vaccine intramuscularly, into the filet of the fish.

The machines handle fish in the sizes from 120 mm to 250 mm (20-150 grams). Once finished, it sorts the vaccinated fish into three different sizes. In addition, it has a channel for misplaced, undersized or rejected fish.

On the other end of the spectrum, the vaccine may be administered manually via a syringe containing one or several needles. See, e.g., MICRO-MATIC® syringe sold by Pharmaq. This product comes in single and double size. The single syringe is available in two sizes: 0.05 ml per dose and 0.1 ml per dose. Additionally, the 0.05 ml syringe can be supplied with an interchangeable 0.025 ml piston if required. The double syringe is available in three different dose size combinations: 0.05 ml+0.05 ml per dose; 0.05 ml+0.1 ml per dose; and 0.1 ml+0.1 ml per dose. The 0.05 ml syringe can be supplied with an interchangeable 0.025 ml piston if required. These syringes can be used for both water-based and oil-based vaccine formulations. The dose size can easily be adjusted+/−10%. The dose accuracy is documented to deviate less than 3%.

The pressure to deliver the injection is not important and may be derived from hydraulic, pneumatic, electrical, or mechanical sources.

The DNA vaccines according to any embodiments disclosed above are administered to salmonids intramuscularly. Muscles suitable for administration of the DNA vaccines disclosed herein include, without limitations skeletal muscle (the entire fish fillet) and epaxial muscle (the muscle on the upper half of the fish).

### Second Vaccine

The DNA vaccine according to any of the embodiments recited above is intramuscularly administered substantially simultaneously with a second vaccine, which is formulated as a water-in-oil (W/O) injection and administered intraperitoneally.

Multiple antigens suitable for use in the second vaccine are known and are already commercially available. In addition, representative isolates of relevant fish pathogens are available from various sources.

In particular embodiments of the invention said antigen from a bacterial source is selected from the group consisting of killed bacteria of the species Moritella viscosa, Piscirickettsias sp. Aeromonas sp., Vibrio sp., Aliivibrio sp., Listonella sp., Tenacibaculum sp., Pasteurella sp., Photobacterium sp, Flavobacterium sp., Yersinia sp., Renibacterium sp., Streptococcus sp., Lactococcus sp., Leuconostoc sp., Bifidobacterium sp., Pediococcus sp., Brevibacterium sp., Edwarsiella sp., Francisella sp., Pseudomonas sp., Cytophaga sp., Nocardia sp., Mycobacterium sp., parts or subunits of these bacteria, and any combination hereof.

Isolates of such bacteria are available, e.g. from LGC Promochem/American Type Culture Collection ATCC repository and distribution center (ATCC) including strains of A. salmonicida (ATCC 33658), V. salmonicida (ATCC 43839), V. anguillarum serotype O1(ATCC 43305) and O2(ATCC 19264). In addition, cultures of Piscirickettsias salmonis have been deposited in the European Collection of Cell Culture (ECACC), Health Protection Agency, Porton Down, Salisbury, Wiltshire (UK), SP4 0JG UK on the 9 Jun. 2006 under the following accession numbers: 06050901, 06050902, 06050903 and 07032110.

Other specific embodiments pertain to a vaccine, wherein said antigenic material obtained from a viral source other than the fish virus as defined above is from a virus selected from the group consisting of: Viral Hemorrhagic Septicemia Virus (VHSV), Infectious Hematopoietic Necrosis virus (IHNV), Infectious Pancreatic Necrosis Virus (IPNV), Infectious Salmon Anaemia virus (ISAV), Salmon pancreatic disease virus (SPDV), Iridovirus, Nodavirus, Piscine myocarditis virus (PMCV) and Piscine Orthoreovirus (PRV) which causes Heart and Skeletal Muscle Inflammation (the virus is sometimes referred to as HSMIV or HSMI virus). These antigens may be included as modified live or inactivated organisms, as parts or subunits of any one of these viruses, as DNA vaccines, and/or combinations thereof. Representative species of such viruses are available to the skilled artisan, for instance from the following deposits: infectious pancreatic necrosis virus (IPNV, ATCC VR-1318, country of origin: unknown), Viral Hemorrhagic Septicemia Virus (VHSV, ATCC VR_1389, country of origin: Denmark); Infectious Hematopoietic Necrosis virus (IHNV, ATCC VR-1392, country of origin: USA)); Infectious Pancreatic Necrosis Virus; Infectious Salmon Anaemia (ISA) virus (ATCC VR-1554, country of origin: Canada). Patent deposits have previously been made by the present applicant of the following viral species: PRV (patent deposit nr ECACC 04050401, country of origin: Norway).

In more specific embodiments, said antigenic material is a subunit that may be selected is from the group consisting of: Glycoprotein of Viral Hemorrhagic Septicemia Virus (VHSV), nucleoprotein of Viral Hemorrhagic Septicemia Virus (VHSV), glycoprotein of Infectious Hematopoietic Necrosis virus (IHNV), nucleoprotein structural proteins of Infectious Pancreatic Necrosis Virus (IPNV), antigenic fragments of any of one of these proteins and combinations hereof.

In other embodiments said antigenic material from an additional parasitic source is from a source selected from the Lepeophtheirus Sp., Caligus Sp., and Ichthyophthirius Sp, parts of any one of these parasites, and combinations thereof. In yet other embodiments said antigenic material is from a fungal source selected from the group consisting of Saprolegnia Sp., Branchiomyces sanguinis, Branchiomyces demigrans and Icthyophonus hoferi.

In certain embodiments, the antigens to be included into the second vaccine are selected form the group consisting of IPNV, ISAV, SPDV, Aeromonas salmonicida, Vibrio anguillarum O1, O2, Vibrio (Aliivibrio) salmonicida, Yersinia ruckeri O1 and Moritella viscosa.

In other embodiments, the antigens in the second vaccine are selected from the group consisting of IPNV, M. viscosa, Aeromonas salmonicida, Vibrio anguillarum serotype 1 and O2, and Vibrio (Aliivibrio) salmonicida.

Suitable vaccines are commercially available and include, without limitations ALPHA JECT® micro 6, ALPHA JECT Micro® 7 ILA/ISA, ALPHA JECT Micro® 1 PD, ALPHA ERM SALAR, ALPHA JECT® Micro 4-2, and ALPHA JECT® 5-1.

The vaccines of the invention (including both the DNA vaccine and the second vaccine) may further comprise a suitable pharmaceutical carrier and/or an adjuvant. The pharmaceutical carriers can be sterile liquids, such as water or buffer solutions, such as saline solutions and aqueous dextrose and glycerol solution. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin. The formulation should suit the mode of administration.

Preferably, the second vaccine is administered by an injection in a microdose such that the volume of one dose is under 500 μl, or under 400 μl, or under 300 μl or under 200 μl or about 100 μl or under 100 μl, or about 50 μl or about 25 μl.

In certain embodiments, the uses described herein do not include the substantially simultaneous administration of a DNA vaccine encoding a Salmon pancreas Disease virus antigen, whether as a separate vaccine or in the same vector as the PMCV DNA vaccine described herein.

In other embodiments, the uses described herein do not include the administration of a DNA vaccine encoding a Pancreatic Disease virus antigen and the substantially simultaneous co-administration of the DNA vaccine encoding the PMCV antigen and the second vaccine as recited above, within several days of each other. Thus, the DNA vaccine encoding the PMCV antigen and the DNA vaccine encoding the Pancreatic Disease virus antigen are not administered within about 1 day, about 3 days, about 5 days, about 7 days, about 10 days, about 12 days, about 14 days, about 16 days, about 18 days, about 20 days or about 21 days of each other. In other words, the DNA vaccine encoding the PMCV antigen and the DNA vaccine encoding the Pancreatic Disease virus antigen may be administered at least about 1 day apart, or about 3 days, about 5 days, about 7 days, about 10 days, about 12 days, about 14 days, about 16 days, about 18 days, about 20 days or about 21 days apart.

In yet other embodiments, the uses described herein do not include the administration of a DNA vaccine encoding a Pancreatic Disease virus antigen at the same site as the administration of the DNA vaccine containing the PMCV antigen according to the embodiments recited herein. In this set of embodiments, the injection site for the DNA vaccine encoding the PD antigen and the injection site for the DNA vaccine encoding the PMCV antigen should be at least 3 mm apart, or about 5 mm apart, or about 7 mm apart, or about 9 m apart, or about 10 mm apart, or about 12 mm apart or about 14 mm apart or about 16 mm apart or about 18 mm apar tor about 20 mm apart or even a greater distance from each other.

The vaccine disclosed herein may be used in protecting multiple salmonid species against an infection. Suitable salmonids include, without limitations, Atlantic salmon (Salmo salar), coho salmon (Oncorhynchus kisutch), rainbow trout (Oncorhynchus mykiss), sockeye salmon (Oncorhynchus nerka), Chinook salmon (Oncorhynchus tshawytscha) and other species.

Salmonids of different ages (or weights) may be vaccinated according to the invention. In certain embodiments, the salmonid weighs between about 15 and about 200 grams at the time of vaccination. Thus, the weight of the salmonid at the time of the vaccination may be between about 25 and about 150 grams or between about 40 and about 110 grams or between about 50 and about 100 grams.

The inventors have surprisingly discovered that if the salmonids treated with the vaccines described herein are not subjected to a smoltification signal within one week (or more) after the vaccination, the immune response to PMCV is further improved. Thus, in certain embodiments the fish are not subjected to smoltification within at least 1 week of the vaccination, or at least 10 days, or at least 14 days, or at least 18 days or at least 21 days, or at least 28 days, or at least 30 days, or at least 35 days or at least 42 days or even longer, after the vaccination according to the methods disclosed herein. Accordingly, in more specific embodiments, the salmonids treated with the vaccines according to the use of any embodiment recited herein are not subjected to a smoltification signal within six weeks after the vaccination with the vaccines disclosed herein.

Multiple smoltification signals are known in the art. Without limitations, the smoltification signal may be selected from the group consisting of light, feed, water temperature and any combination thereof. See, e.g., EP 3197290A1.

The invention will now be described in the following illustrative examples.

### EXAMPLES

### Example 1

Atlantic salmon (n=240) with mean weight of 24 g were kept in four 150 L tanks (n=60 per tank) with freshwater (12C). The 60 fish in each tank were starved for one day, before they were anaesthethized using MS222 (Tricain, PHARMAQ AS) and then tagged and vaccinated with one of four different vaccination regimes (Table 1). The vaccine-regimes included fish vaccinated with CMS DNA-vaccines alone or in combination with the multivalent commercial vaccine ALPHA JECT® micro 6 (PHARMAQ AS) (n=15 per group per tank). ALPHA JECT® micro 6 (AJm6) is a water-in-oil emulsion containing the following inactivated antigens: Aeromonas salmonicida subsp. salmonicida, Listonella anguillarum (Vibrio anguillarum) serotypes O1 and O2, Vibrio salmonicida, Moritella viscosa, and Infectious Pancreatic Necrosis Virus.

The CMS DNA vaccine had the NTC9385R NANOPLASMID™ backbone into which the nucleic acid sequence encoding PMCV antigen having the amino acid sequence of SEQ ID NO: 3 and the nucleic acid sequence encoding either IFNb having the amino acid sequence of SEQ ID NO: 7, or IFNb1 having the amino acid sequence of SEQ ID NO: 9 were subcloned, each under operative control of CMV promoter.

Administration of the DNA-vaccines were done by giving two injections of 0.05 ml intramuscularly into skeletal muscle (one on each side), 10 μg of the plasmid per injection, while ALPHA JECT® micro 6 was given as one injection of 0.05 ml intraperitoneally. Groups that received both the DNA-vaccine and ALPHA JECT® micro 6 were vaccinated under the same anaesthethic period.

Two of the tanks were smoltified by giving the fish a 24:0 light:dark light-regime for 6 weeks after administration of the vaccines. Following these 6 weeks, the fish in these two tanks were transferred to seawater and a 12:12 light:dark regime. The remaining two tanks were not smoltified, and kept on a 12:12 light:dark regime and freshwater the whole study period. The experimental setup is summarized in Table 1.

Following a 48 days immunization period, all groups were anaesthethized and challenged with PMCV by intraperitoneal injection (0.1 ml) of a tissue homogenate containing infectious PMCV (isolate ID AL V1289) originating from a Norwegian field outbreak of CMS. Two of the tanks (one freshwater and one seawater) were terminated and sampled 3 weeks after challenge, while the remaining two tanks were sampled 7 weeks after challenge. For the first sampling point, heart and kidney were sampled on RNAlater® for subsequent RNA extraction and quantification of viral RNA using a commercially available PMCV specific real-time PCR assay (PHARMAQ Analytiq AS). This was repeated for the last sampling point, but with the addition of heart sampling on formalin for histological analysis and semi-quantitative scoring of heart lesions according to severity, where 0 indicates no pathologic finding and 3 indicate presence of severe pathology (service provided by PHARMAQ Analytiq, Norway).

TABLE 1

| | | | Dose | Dose | | | | |
|---|---|---|---|---|---|---|---|---|
| | Vaccine | Vaccine | vaccine | vaccine | | Smolti- | | No. |
| Group | 1 | 2 | 1 - IP (ml) | 2 - IM (ml) | Tank | fication | Tag | fish |
| 1 | AJm6 | CDC PMCV- | 0.05 | 2 × 0.05 | C7K1 | Yes | None | 15 |
| | | IFNb | | | | | | |
| 2 | AJm6 | — | 0.05 | — | C7K1 | | AF | 15 |
| 3 | — | PMCV-IFNb | — | 2 × 0.05 | C7K1 | | RM | 15 |
| 4 | — | PMCV-IFNb1 | — | 2 × 0.05 | C7K1 | | LM | 15 |
| 1 | AJm6 | PMCV-IFNb | 0.05 | 2 × 0.05 | C7K2 | Yes | None | 15 |
| 2 | AJm6 | — | 0.05 | — | C7K2 | | AF | 15 |
| 3 | — | PMCV-IFNb | — | 2 × 0.05 | C7K2 | | RM | 15 |
| 4 | — | PMCV-IFNb1 | — | 2 × 0.05 | C7K2 | | LM | 15 |
| 1 | AJm6 | PMCV-IFNb | 0.05 | 2 × 0.05 | C7K3 | No | None | 15 |
| 2 | AJm6 | — | 0.05 | — | C7K3 | | AF | 15 |
| 3 | — | PMCV-IFNb | — | 2 × 0.05 | C7K3 | | RM | 15 |
| 4 | — | PMCV-IFNb1 | — | 2 × 0.05 | C7K3 | | LM | 15 |
| 1 | AJm6 | PMCV-IFNb | 0.05 | 2 × 0.05 | C7K4 | No | None | 15 |
| 2 | AJm6 | — | 0.05 | — | C7K4 | | AF | 15 |
| 3 | — | PMCV-IFNb | — | 2 × 0.05 | C7K4 | | RM | 15 |
| 4 | — | PMCV-IFNb1 | — | 2 × 0.05 | C7K4 | | LM | 15 |

AF—Adipose Fin;
LM—Left Maxilla,
RM—Right Maxilla

At both sampling-points, blood was sampled on heparin coated vacutainers from the groups that had been vaccinated with ALPHA JECT® micro 6. Blood plasma was subsequently used in an ELISA analysis to quantify the amount of IgM against Aeromonas salmonicida (one of the components in ALPHA JECT® micro 6). Local reactions caused by ALPHA JECT® micro 6 was also assessed from these fish using a modified Speilberg's scale.

TABLE 2
Real-time PCR results from hearts of fish challenged
with PMCV 3 weeks earlier. Negative fish were
given a Ct-value of 40 by default.

| | | | | Number of | Mean ct |
|---|---|---|---|---|---|
| | | Water | Fish | PMCV positive | value |
| Group | Vaccines | salinity | (n) | fish | (PMCV) |
| 1 | AJm6 + | SW | 15 | 15 | 29.2 |
| | PMCV-IFNb | | | | |
| 2 | AJm6 | SW | 15 | 15 | 22.9 |
| 3 | PMCV-IFNb | SW | 15 | 15 | 27.1 |
| 4 | PMCV-IFNb1 | SW | 15 | 4 | 37.4 |
| 1 | AJm6 + | FW | 15 | 14 | 25.5 |
| | PMCV-IFNb | | | | |
| 2 | AJm6 | FW | 15 | 15 | 22.3 |
| 3 | PMCV-IFNb | FW | 15 | 12 | 23.7 |
| 4 | PMCV-IFNb1 | FW | 15 | 4 | 32.5 |

TABLE 3
Histological analysis of heart atriums. Histological lesions
were scored from 0 (normal) to 3 (severe pathology). The number
of fish given each score for each group is indicated.

| | | Water | Mean | Distribution of scores | | | | | | |
|---|---|---|---|---|---|---|---|---|---|---|
| Group | Vaccines | salinity | score | 0 | 0.5 | 1 | 1.5 | 2 | 2.5 | 3 |
| 1 | AJm6 + | SW | 1.07 | 5 | 1 | 4 | 0 | 3 | 1 | 0 |
| | PMCV-IFNb | | | | | | | | | |
| 2 | AJm6 | SW | 2.80 | 0 | 0 | 0 | 0 | 2 | 2 | 11 |
| 3 | PMCV-IFNb | SW | 1.03 | 7 | 0 | 3 | 0 | 2 | 1 | 2 |
| 4 | PMCV- | SW | 1.04 | 2 | 4 | 3 | 1 | 2 | 0 | 1 |
| | IFNb1 | | | | | | | | | |
| 1 | AJm6 + | FW | 1.40 | 2 | 3 | 4 | 1 | 0 | 2 | 3 |
| | PMCV-IFNb | | | | | | | | | |
| 2 | AJm6 | FW | 2.93 | 0 | 0 | 0 | 0 | 0 | 2 | 13 |
| 3 | PMCV-IFNb | FW | 0.58 | 6 | 2 | 2 | 1 | 0 | 1 | 0 |
| 4 | PMCV- | FW | 0.57 | 9 | 1 | 3 | 0 | 1 | 0 | 1 |
| | IFNb1 | | | | | | | | | |

TABLE 4
Local reactions (adhesions, modified Speilberg's score) and IgM-
titers against Aeromonas salmonicida 14 weeks after vaccination.

| | | | | Adhesion | IgM-titer |
|---|---|---|---|---|---|
| | | Water | Fish | score | (geometric |
| Group | Vaccines | salinity | (n) | (mean) | mean) |
| 1 | AJm6 + | SW | 11 | 1.37 | 45897 |
| | PMCV-IFNb | | | | |
| 2 | AJm6 | SW | 15 | 1.87 | 48170 |
| 1 | AJm6 + | FW | 15 | 1.37 | 60690 |
| | PMCV-IFNb | | | | |
| 2 | AJm6 | FW | 15 | 1.47 | 63560 |

The results demonstrated that co-administration of a multivalent vaccine ALPHA JECT micro 6 did not impair the protection provided by the CMS-vaccine under the smoltification regime. Local reactions caused by ALPHA JECT® micro 6 were the same or lower in co-administered groups (p<0.05 in saltwater, p>0.05 in fresh water, using Mann-Whitney test), and only a slight, statistically insignificant reduction (p>0.05, using a Mann-Whitney test) in IgM-titers against A. salmonicida were observed following coadministration.

This suggests that co-administration of a DNA-vaccine against CMS and a multivalent core vaccine only has limited interactions, that are unlikely to affect the performance of any of the vaccines. Surprisingly, co-administration of the DNA vaccine and ALPHA JECT® micro 6 slightly increased viral count (i.e., reduced viral load) in both the fresh water group and the sea water group (see Table 2, comparison between group 1 and 3 in the fresh water thank and in the seawater tank).

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of protecting a salmonid against Piscine Myocarditis virus (PMCV) and at least one other pathogen, said method comprising administering to said salmonid a DNA vaccine comprising a nucleic acid sequence encoding SEQ ID NO: 3 or SEQ ID NO: 4 or an amino acid sequence at least 90% identical to SEQ ID NO: 3 or NO: 4 DNA vaccine comprising a nucleic acid sequence encoding SEQ ID NO: 3 or SEQ ID NO: 4 or an amino acid sequence at least 90% identical to SEQ ID NO: 3 or NO: 4 wherein
a) said DNA vaccine is injected intramuscularly
b) a second vaccine is injected intraperitoneally substantially simultaneously with the DNA vaccine;
c) said second vaccine comprises an antigen that elicits protective response against said at least one other pathogen; and
d) said second vaccine is formulated as a W/O emulsion.

2. The method of claim 1, wherein the nucleic acid sequence encodes SEQ ID NO: 5 or an amino acid sequence at least 90% identical thereto.

3. The method of claim 2, wherein the nucleic acid sequence encodes SEQ ID NO: 5 or an amino acid sequence at least 95% identical thereto, and wherein further at least 50% of the amino acids different from SEQ ID NO: 5 are conservative substitutions

4. The method of claim 1 wherein the nucleic acid sequence encodes SEQ ID NO: 5.

5. The method of claim 1 further comprising a molecular adjuvant.

6. The method of claim 5 wherein said molecular adjuvant is IFNb1.

7. The method of claim 5 wherein said molecular adjuvant is at least 95% identical to SEQ ID NO: 7 or to SEQ ID NO: 9.

8. The method of claim 7, wherein said molecular adjuvant is a conservatively substituted variant of SEQ ID NO: 7 or SEQ ID NO: 9.

9. The method of claim 7, wherein the molecular adjuvant comprises SEQ ID NO: 7 or SEQ ID NO: 9.

10. The method of claim 9 wherein the molecular adjuvant is encoded by a nucleic acid sequence that is identical to SEQ ID NO: 8 or SEQ ID NO: 10.

11. The method of claim 1, wherein said DNA vaccine is administered in two or three intramuscular injections, said intramuscular injections administered substantially simultaneously and wherein the total dose of the molecular adjuvant in said DNA vaccine administered in two or three injection is no greater than the dose of the molecular adjuvant in said DNA vaccine administered in a single injection.

12. The method of claim 4, wherein said DNA vaccine is administered in two or three intramuscular injections, said intramuscular injections administered substantially simultaneously and wherein the total dose of the antigen in said DNA vaccine administered in two or three injection is no greater than as the dose of the antigen in said DNA vaccine administered in a single injection.

13. The method according to claim 1, wherein said second vaccine comprises one or more of Moritella viscosa, Piscirickettsias sp. Aeromonas sp., Vibrio sp., Aliivibrio sp., Listonella sp., Tenacibaculum sp., Pasteurella sp., Photobacterium sp, Flavobacterium sp., Yersinia sp., Renibacterium sp., Streptococcus sp., Lactococcus sp., Leuconostoc sp., Bifidobacterium sp., Pediococcus sp., Brevibacterium sp., Edwarsiella sp., Francisella sp., Pseudomonas sp., Cytophaga sp., Nocardia sp., Mycobacterium sp., Viral Hemorrhagic Septicemia Virus (VHSV), Infectious Hematopoietic Necrosis virus (IHNV), Infectious Pancreatic Necrosis Virus (IPNV), Infectious Salmon Anaemia virus (ISAV), Salmon pancreatic disease virus (SPDV), Iridovirus, Nodavirus, Piscine myocarditis virus (PMCV) and Piscine Orthoreovirus (PRV).

14. The method according to claim 1, wherein said second vaccine comprises one or more of IPNV, ISAV, SPDV, Aeromonas salmonicida, Vibrio anguillarum O1, O2, Vibrio (Aliivibrio) salmonicida, Yersinia ruckeri O1 and Moritella viscosa.

15. The method according to claim 14, wherein said second vaccine comprises IPNV, M. viscosa, Aeromonas salmonicida, Vibrio anguillarum serotype 1 and O2, and Vibrio (Aliivibrio) salmonicida.

16. The method according to claim 1, wherein said salmonid is Atlantic salmon (Salmo salar)

17. The method according to claim 1, wherein said salmonid weighs about 40 to about 110 grams.

18. The method according to claim 1 wherein said vaccine reduces the frequency, the intensity, or the duration of at least one clinical sign selected from the group consisting of mortality, lack of weight gain, skin ulcers, and a heart defect.

19. The method according to claim 1, wherein said salmonid is not subjected to a smoltification signal within at least one week of injecting said DNA vaccine.

20. The method according to claim 1, wherein said salmonid is not subjected to a smoltification signal within six weeks of injecting said DNA vaccine.

21. (canceled)


SEQUENCE LISTING (12 sequences)

SEQ ID NO: 1 - protein, 20 aa, synthetic construct
MEWNTFFLVILIIIIKSTTP

SEQ ID NO: 2 - protein, 21 aa, synthetic construct
VGGAFLLLVLCCCCKASPPIP

SEQ ID NO: 3 - protein, 861 aa, Piscine Myocarditis virus
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 4 - protein, 666 aa, synthetic construct
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRGGGGSAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 5 - protein, 779 aa, synthetic construct
MEWNTFFLVILIIIIKSTTPQITQRPPVENISTYHADWDHHHHHHGGGSGGRMEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRGGGGSAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDIGGRNLIPSDWSFHWSIWPSLSGMGVVGGAFLLLVLCCCCKASPPIPNYGIPMQQFSRSQTV

SEQ ID NO: 6 - DNA, 2340 bp, synthetic construct
  source 1..2340: mol_type = other DNA
atggaatggaacacttttttcttggtgatcttgatcatcatcataaagagcaccacaccacagatcactcaacgacctccggtcgaaaacatctcgacgtaccatgcagattgggaccatcatcaccatcaccacggtggcggttcgggcggccgcatggaaccaaacacatctgtcattgcaacggagcagcagcaggctgccatgagagaggtggaggccgaggcggcggccagagacgaagtggtggagaagatcgcattcgctgaaggagcgatgatggtacagacgaggaggttaccatcaggaaagtcgtcggtaggaggttttctcggcgaactggcacagaacatacgtgccatgaatcggtcattgcacacagataccaacatgctgaccgaaggggcgatggtggacagagcgagggcaaaagtacacaaaatcattagggaagggaatttggactctagggtattttcaaacacggggagcaacactatgttgtcactgtgggtaccagcagtaccgggaccaccggcagtaccggagcattgggacgttgcgccgtcctggttcgtatgcagaccggggaaaaagggggggataaagatcacacaaagcgcatcaatggcagcattaaacccactatttagaggcgcagacgtggggccaatcgggacagcagtcagggcggatgtaaacgcattttcaatgaatgcagttctgggagcactaagagccgggggatttaacaccgaacattccctggtgtcattcgttgaaccactaattcggatcttgctaatgggggtacaaacacaagacagggggaccagcccatgggattgggttggagggatgagttcgcgaatagtcaatcccctagtattcacaacaagcgggaacttcttcccagggggaccaaatttgagggtgtggggagccaacgatacagtggccaggatagtaaacgttgaggactacatgcgcgaggcggccggggaggggaggttcgacgctggatggggaccggaattctggggtgggacaggggacgacgcagtggcggtggtaccgataagggcagtagaagcagggctaggagaagtaaacgcagggtggacattggcacacatggaatacccagtcaaggttagactacttgacgtcgacgaccgaacaattggaccaggggggagcctgcccctaaacgcaaacagagaatacacggcggcaggagctacgcatgtacccgggccctatgccagggtactgtacgtcgtcgtggaccaaaacgcagacaggtgtgtgggggtgagagtgcagggacagggtgctgtaattgacgtggacccggcgttgaattacgtgatagggggagcggatttggggatgttgccgttgatacagtggagtgtagggctgggggccgaggacatggcgcagggatcgattgcacagacgcagcgatgggtgaggatgtatggaaacgaggacgattgggaatcagcgtggcatctagtgtctagcgcgtacacagtgtacagcccggcattcaggagatcgggtgtcgcagtggagggaggattctgggcgcaaccagctgcaggggcagcaccgtttccactaggaggattggcagggtgggtgaggggtggcggtggctcggcggtagaggggatggcaggtgcgcaggggggtatctgggacgtggtccagtaccagctgcctgggcctgacgatgaggcgaggggggtgatgaacacggtgggggcgatggggggatggacgagggcggtgacaccagtagacaatgtggccaccatgagggacaacggggttgagggggaaccttgtggaatagtgatgtctctaccaacaagtgggaccgctgtggtggataggttagctaatttcggattaccaccagcgagggcggaattaagagaagtaccatttggcgggtaccaaagatcagtcacaaacaccaaccacagagtcaaggtgagtgtgagtggggggcgagcagttgttcaaaaagggaacaaagccgagatgaatccagtctttgtcaataggacaccaggacaaacgaccctaggccaaccaacaacagacactacagggatgacaactgcagattttttagatataggcggccgcaacctaatcccttcggattggtcattccactggagtatttggccctcattatctgggatgggggttgtgggaggggccttccttctactggtactctgctgttgctgcaaggcgtcccctcccattccaaattacgggattccgatgcagcagttctccagaagtcagacggtctga

SEQ ID NO: 7 - protein, 183 aa, Salmo salar
MAVLKWLSICLTLFCQGTAASKPCRWTQFRLGKLNDVSIGLLSDMGGLFPLMCAEESVEQMFPEDLYKNTEGEDVYVVALEAMRYVEQLYNNSLTSVTWNKTKLNMFQNVIYRQVQNLELCVVGGVWESSGDGWSVTLKTYFNKLNTVLKEKEHSACAWEIVRKEIRENLVQFKKFIDSRVKL

SEQ ID NO: 8 - DNA, 552 bp, synthetic construct
  source 1..552: mol_type = other DNA
atggcagtactgaagtggctttccatctgcctgactctgttctgccaaggaaccgcggcctcaaagccttgtcggtggacccagttccggctggggaaactgaatgacgtttcaattggcctactctccgacatggggggactcttcccgctgatgtgcgccgaggagtcggtggagcagatgtttccagaggacctctacaagaacaccgagggcgaagatgtgtacgtggttgctctggaagccatgcgttacgtagagcagctgtacaataactcacttacctccgtcacctggaacaaaactaagctcaacatgttccagaacgttatctacagacaggtgcagaaccttgagctatgcgttgtaggcggggtttgggagtctagtggtgacggatggtccgttacacttaagacctactttaataaactgaacactgtgctgaaggagaaagagcatagcgcttgtgcctgggagattgtccgcaaggagattagggagaacctggttcagtttaagaagtttatcgactcaagagtgaaactgtga

SEQ ID NO: 9 - protein, 183 aa, Salmo salar
MAVLKWLSICLTLFCQGTVASKPCRWTQFRLGKLNDVSIGLLSDMGGLFPLMCAEENVEQMFPEDLYKNTEGEDVSVVALEAMRYVEQLYNNSLTSATWSKTKLNMFQNVIYRQVQNLELCVVGGVWESSGDGWSVTLKTYFNKLNTVLKEKEYSACAWEIVRKEIRENLVQFKKFIDSRVKL

SEQ ID NO: 10 - DNA, 552 bp, synthetic construct
  source 1..552: mol_type = other DNA
atggctgtactgaaatggctgtccatctgtctgacactgttctgccagggaactgtagcttcaaagccatgcaggtggactcagttccgcttggggaaactaaatgacgtgagcatcggtttgctgtccgacatgggaggactgtttccactgatgtgtgctgaggagaacgtggagcagatgttccccgaggacctgtataagaacaccgagggcgaggacgtgtccgtcgtggccctggaggcgatgcggtacgtcgagcaactttacaacaactcgctcacttccgccacatggagtaagacgaagctcaacatgttccagaacgtgatctacagacaggtgcagaacctagagctgtgtgtggtggggggggtatgggagtccagtggggatggctggtccgttaccctgaagacatactttaacaagcttaataccgtgctgaaagagaaagaatactcggcgtgtgcctgggagattgtgcggaaggaaatcagggaaaatctggtgcagttcaagaaattcattgactcaagggtaaagttgtga

SEQ ID NO: 11 - protein, 39 aa, synthetic construct
MEWNTFFLVILIIIIKSTTPQITQRPPVENISTYHADWD

SEQ ID NO: 12 - protein, 58 aa, synthetic construct
NLIPSDWSFHWSIWPSLSGMGVVGGAFLLLVLCCCCKASPPIPNYGIPMQQFSRSQTV